(19) United States
(12) Patent Application Publication
(10) Pub. No.: US 2018/0344928 A1
(43) Pub. Date: Dec. 6, 2018

(54) FLOWRATE REGULATING DEVICE FOR INFUSION SET

(71) Applicant: Hyun-Woo LEE, Hanam-si (KR)

(72) Inventor: Hyun-Woo LEE, Hanam-si (KR)

(21) Appl. No.: 15/774,640

(22) PCT Filed: Sep. 29, 2016
(86) PCT No.: PCT/KR2016/010869

§ 371 Date: May 9, 2018

(30) Foreign Application Priority Data
Nov. 26, 2015 (KR) 10-2015-0166303
Jul. 22, 2016 (KR) 10-2016-0093116

(51) Int. Cl. A61M 5/168; A61M 39/22

(52) U.S. Cl. CPC A61M 5/16881; A61M 2039/229; A61M 39/22

(57) ABSTRACT

The present invention relates to a flowrate regulating device for a medical infusion set and, more specifically, to a flowrate regulating device for an infusion set, the device comprising: a body member formed in a cylindrical shape opened vertically, and having an inflow tube and a discharge tube, which protrude from both sides thereof; a rotating member rotatably coupled to the upper end of the body member, and having a plurality of flowrate regulating passages provided on the outer diametral surface thereof and connected to the inflow tube; a fixing member fixedly coupled to the lower end of the body member, and having a discharge passage provided on the upper surface thereof and connecting the lower ends of the flowrate regulating passages and the discharge tube; and an injection port unit selectively provided at the rotating member and/or the fixing member.

### TECHNICAL FIELD

The present invention relates to a flowrate regulating device for a medical infusion set, and more particularly, to a flowrate regulating device for an infusion set, which includes: a body member formed in a cylindrical shape opened vertically and having an inflow tube and a discharge tube, which respectively protrude from both sides thereof; a rotating member rotatably coupled to the upper end of the body member and having a plurality of flowrate regulating passages provided on the outer circumferential surface thereof and connected to the inflow tube; a fixing member fixedly coupled to the lower end of the body member and having a discharge passage provided on the upper surface for connecting the lower ends of the flowrate regulating passages with the discharge tube; and an injection port unit selectively provided on at least one of the rotating member and the fixing member.

### BACKGROUND ART

In general, an infusion set is a medical appliance used for injecting infusion into a human body. FIG. 1 shows a normal infusion set. In FIG. 1, the infusion set includes: a drawing chamber 100 for drawing infusion stored in an infusion bag 10; an infusion tube 200 through which the infusion pass, a connection tube 300 mounted at an end portion of the infusion tube 200; a flowrate regulating clamp 400 and an Y-shaped branch tube 500 mounted in the middle of the infusion tube 200; and a syringe 600 combined to the connection tube 300.

Here, the flowrate regulating clamp 400 functions to regulate a flowrate of infusion flowing into the human body through the infusion tube 200, and includes a guide rail 410 and a pressure roller 420. The conventional flowrate regulating clamp 400 presses the infusion tube 200 while moving the pressure roller 420 on the guide rail 410, so it is difficult to precisely and accurately regulate the flowrate.

In order to solve the above problem, Korean Patent Nos. 10-0468222 (Jan. 17, 2005), 10-0472033 (Feb. 3, 2005), and 10-1487754 (Jan. 23, 2015) disclose rotary type flowrate regulating devices with new structures. Out of them, the medicinal liquid dose amount regulating device 100 disclosed in Korean Patent No. 10-0472033 will be described. The medicinal liquid dose amount regulating device 100 includes a fixing member 10 connected to an infusion tube 300, and a regulating member 20 rotatably combined to the fixing member 10.

The fixing member 10 has an infusion inlet 13 and an infusion outlet 14 disposed at both sides thereof and communicating with the infusion tube 300. The regulating member 20 includes a flow path 23 connected to the infusion inlet 13, a groove 25 connected to the infusion outlet 14, and a through hole 24 for connecting the flow path 23 and the groove 25 with each other. Here, the flow path 23 is formed to be gradually increased in its width and depth as approaching the through hole 24 in a circumferential direction of the regulating member 20 so as to regulate a flow rate of the infusion passing through the flow path 23 according to rotational angles of the regulating member 20.

However, such rotary type flowrate regulating devices have several disadvantages in that it is difficult to do injection molding because the entire structure is very complicated and in that a flow of infusion is not good because a flow line of the flow path in which the infusion passes is too complicated. Furthermore, the rotary type flowrate regulating devices have several disadvantages in that a branch tube 500 having an injection port 510 must be additionally mounted in the middle of the infusion tube, as shown in FIG. 1, in order to inject other medical fluids to the infusion because the devices have a two-way structure.

### PATENT LITERATURE

### Patent Documents

Patent Document 1: Korean Patent No. 10-0468222 (Jan. 17, 2005)

Patent Document 2: Korean Patent No. 10-0472033 (Feb. 3, 2005)

Patent Document 3: Korean Patent No. 10-1487754 (Jan. 23, 2015)

### DISCLOSURE

### Technical Problem

Accordingly, the present invention has been made in view of the above-mentioned problems occurring in the prior art, and it is an object of the present invention to provide a flowrate regulating device for an infusion set, which has a simple structure so as to be easy in injection molding, facilitates a flow of infusion, and prevents a leak of infusion.

It is another object of the present invention to provide a flowrate regulating device for an infusion set, which allows a user to select a three-way structure or a four-way structure and provides a stopcock function to stop a flow path if necessary.

### Technical Solution

To accomplish the above object, according to the present invention, there is provided a flowrate regulating device for an infusion set including: a body member which is formed in a cylindrical shape opened vertically and has an inflow tube protruding at one side and a discharge tube protruding in the opposite direction from the inflow tube at a position lower than the inflow tube; a rotating member of which the lower part is rotatably combined to the top of the body member, the rotating member including a plurality of flowrate regulating passages, which are vertically formed on the outer peripheral surface of the lower part getting in contact with the body member side by side at regular intervals, and a rotating lever mounted on the top thereof; and a fixing member of which the upper part is fixedly combined to the bottom of the body member, the fixing member including a discharge passage hollowed on the upper surface getting in contact with the bottom surface of the rotating member in a diameter direction, wherein the flowrate regulating passages are arranged such that a cross section area of each flowrate regulating passage gradually increases in the peripheral direction of the rotating member, and as the rotating member rotates, one of the flowrate regulating passages is connected to the inflow tube, the discharge passage is connected between a lower end portion of the flowrate regulating passage, which is connected to the inflow tube, and the discharge tube, and the greatest cross section area of the flowrate regulating passage does not exceed a cross section area of the discharge passage.

Moreover, a central hole (H) is vertically perforated in at least one of the rotating member and the fixing member along a central axis line, and an injection port unit is mounted on the central hole (H).

Furthermore, an angle regulating grooves are respectively mounted at lower end portions of the flowrate regulating passages at regular intervals, and retaining jaws corresponding to the angle regulating grooves are mounted on the inner peripheral surface of the body member, so that the retaining jaws fix a rotational angle to accurately connect one of the flowrate regulating passages to the inflow pipe while getting over the angle regulating grooves one by one when the rotating member is rotated.

### Advantageous Effects

As described above, the flowrate regulating device for the infusion set according to an embodiment of the present invention is easy to do injection molding since having a simple structure, facilitates a flow of infusion, and prevents a leak of infusion.

Additionally, the flowrate regulating device for the infusion set according to an embodiment of the present invention may be selectively configured to have a three-way structure or a four-way structure because the injection port unit is mounted at any one of the rotating member and the fixing member as occasion demands.

In addition, the flowrate regulating device for the infusion set according to an embodiment of the present invention is easy to use and can accurately regulate a flowrate because any one of the flowrate regulating passages is accurately connected to the inflow tube by combination between the angle regulating groove formed on the rotating member and the retaining jaw mounted on the body member.

### DESCRIPTION OF DRAWINGS

FIG. 1 is a view showing a structure of a conventional infusion set.

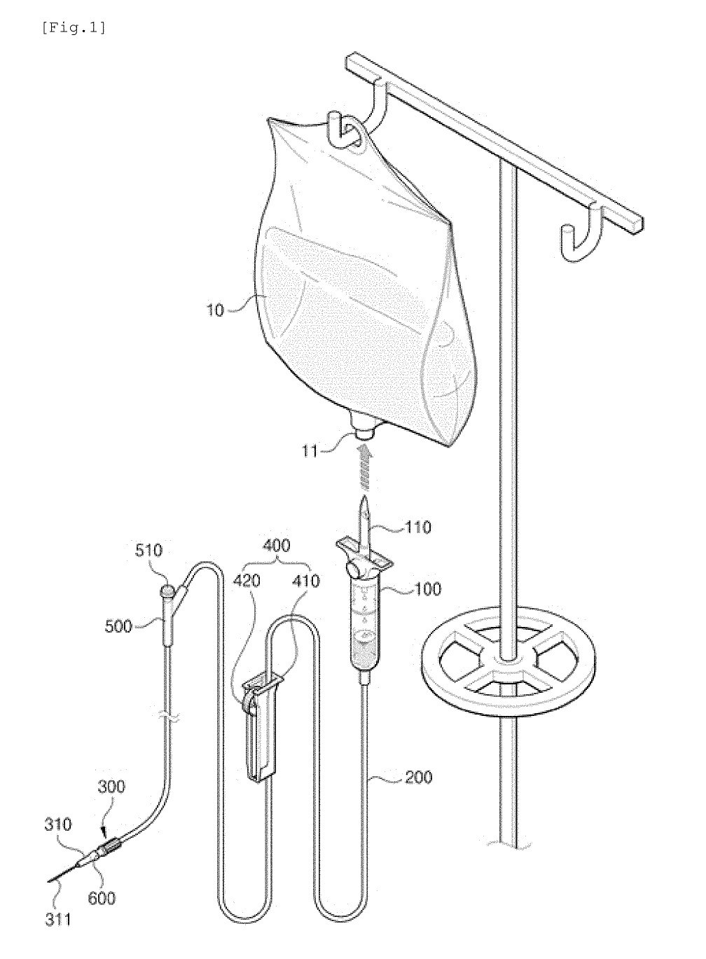

FIG. 2 is a perspective view showing a flowrate regulating device according to a preferred embodiment of the present invention.

FIG. 3 is a sectional view of FIG. 2.

FIG. 4 is a partially exploded perspective view of FIG. 2.

FIG. 5 is a view showing an arranged state of a flowrate regulating passage 21 and an angle regulating groove 23 of FIG. 4.

### EXPLANATION OF ESSENTIAL REFERENCE NUMERALS IN DRAWINGS

| | |
|---|---|
| 10, 10a, 10b: body member | 11: inflow tube |
| 12: outflow tube | 13: retaining jaw |
| 14: first stopper | 15: upper annular groove |
| 16: lower annular groove | 17: fixing groove |
| 20: rotating member | 21: flowrate regulating passage |
| 22: rotational lever | 23: angle regulating groove |
| 24: second stopper | 25: retaining annular jaw |
| 26: flow path shield | 27: free path passage |
| 30: fixing member | 31: discharge passage |
| 32: retaining annular jaw | 33: fixing protrusion |
| 40: injection port unit | 41: insertion tube |
| 42: port housing | 43: rubber stopper |
| 44: end cap | H: central hole |
| S: slit part | |

### MODE FOR INVENTION

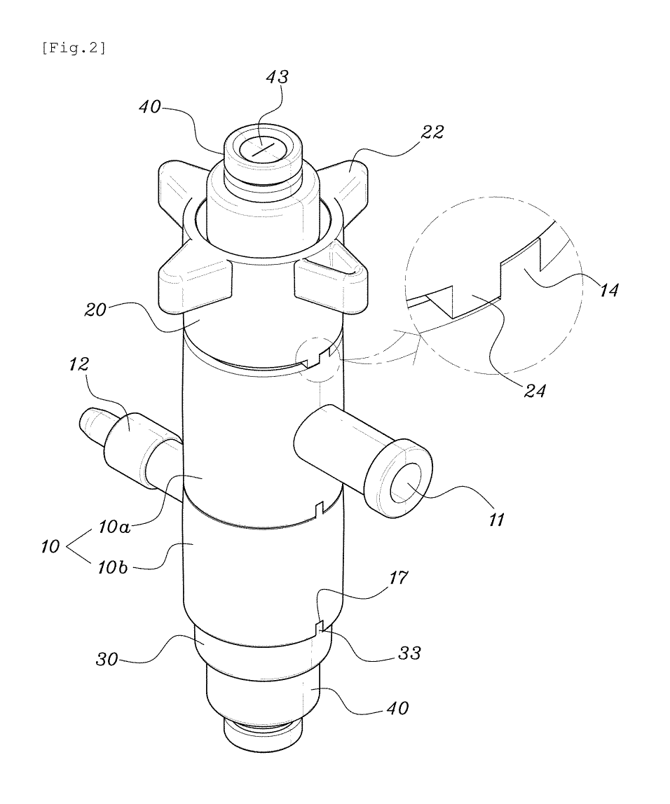

Reference will be now made in detail to a preferred embodiment of the present invention with reference to the attached drawings. However, in spite of essential parts in executing the present invention, in case that the parts belong to known skills or those skilled in the art can easily execute them from the known skills, the detailed description will be omitted. Moreover, for convenience, in FIGS. 2 to 4, based on a body member 10, the part that a rotating member 20 is combined is called an ‘upper’ direction and the part that a fixing member 30 is combined is called a ‘lower’ direction. Furthermore, components having the same function have the same reference numerals even though they are arranged in different locations.

As shown in FIG. 2, a flowrate regulating device for an infusion set according to a preferred embodiment of the present invention includes a body member 10 of a cylindrical shape which is opened in a vertical direction, a rotating member 20 rotatably combined to the top of the body member 10, and a fixing member 30 fixed and combined to the bottom of the body member 10.

First, the body member 10 has an inflow tube 11 and a discharge tube 12, which respectively protrude from both sides thereof. In this instance, preferably, the inflow tube 11 and the discharge tube 12 protrude in opposite directions from each other, and the inflow tube 11 is arranged relatively higher than the discharge tube 12.

The inflow tube 11 is connected with an infusion tube which is connected to, for instance, an infusion bag, and the discharge tube 12 is connected with an infusion tube which is connected to, for instance, a syringe. The connection structure among the inflow tube 11, the discharge tube 12 and the infusion tube may be formed in a forced insertion way or in a screw-coupling way using a tube connector.

The body member 10 may be generally formed in one body or may be divided into an upper body member 10a and a lower body member 10b for the sake of convenient manufacturing. FIGS. 2 to 4 illustrate the case that the body member 10 is divided into two parts, and the inflow tube 11 is mounted on the upper body member 10a and the discharge tube 12 is mounted on the lower body member 10b. The upper body member 10a and the lower body member 10b are combined with each other through a forced fitting way or a screw-coupling way.

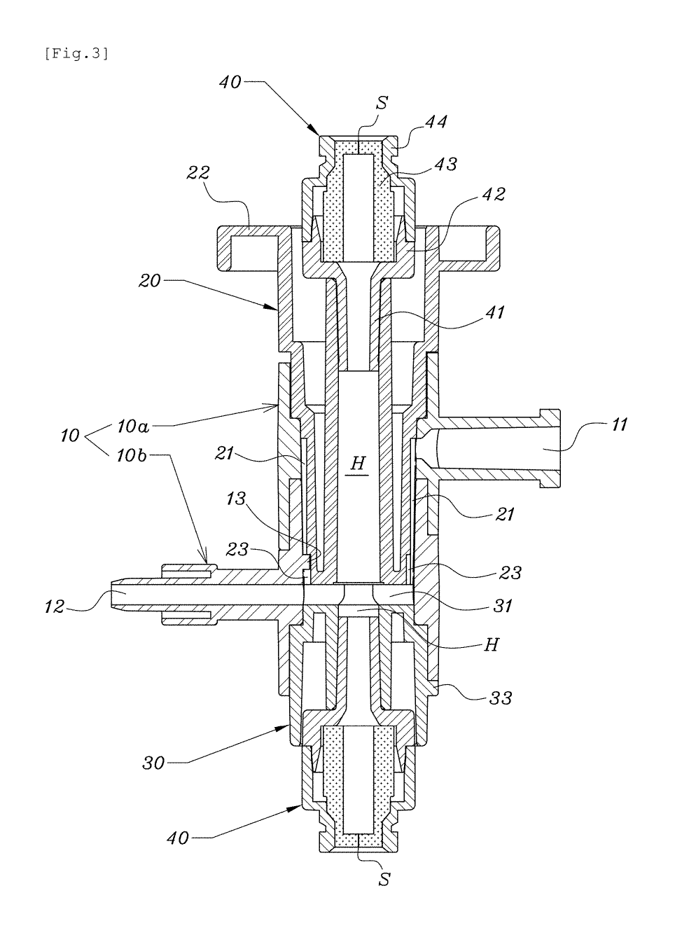

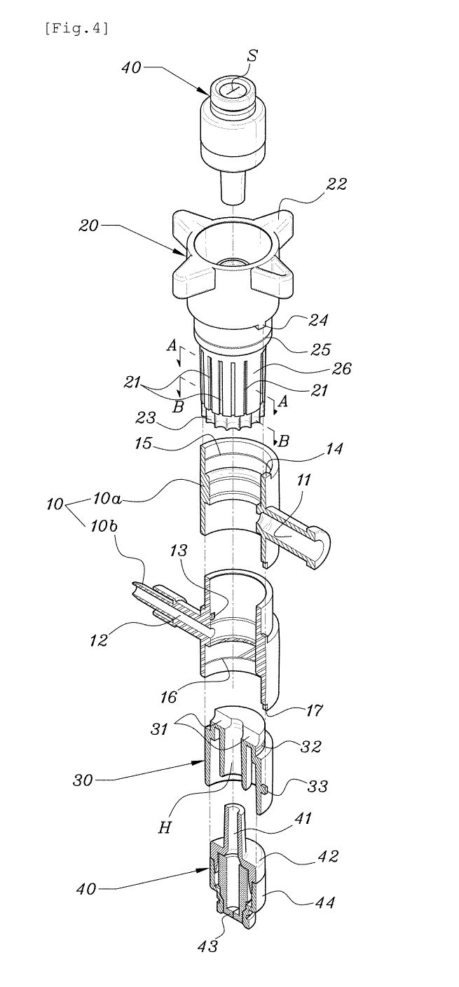

As shown in FIGS. 3 and 4, a lower part of the rotating member 20 is rotatably combined to the top of the body member 10. A plurality of flowrate regulating passages 21, which are formed in the vertical direction, are arranged on the outer peripheral surface of the lower part, gets in contact with the body member 10, side by side at regular intervals.

A rotating lever 22 protrudes on the top of the rotating member 20. Anything will do if the rotating lever 22 has a structure to rotate the rotating member 20 conveniently. One of the flowrate regulating passages 21 is connected to the inflow tube 11 according to a rotational position of the rotating member 20.

An upper part of the fixing member 30 is fixed and combined to the bottom of the body member 10. An upper surface of the fixing member 30 gets in contact with a lower surface of the rotating member 20 inside the body member 10.

The fixing member 30 has a discharge passage 31 formed in the upper surface thereof in a diameter direction as shown in FIGS. 3 and 4. The discharge passage 31 has an end portion connected with a lower end part of the flowrate regulating passage 21 and the other end portion connected to the discharge tube 12.

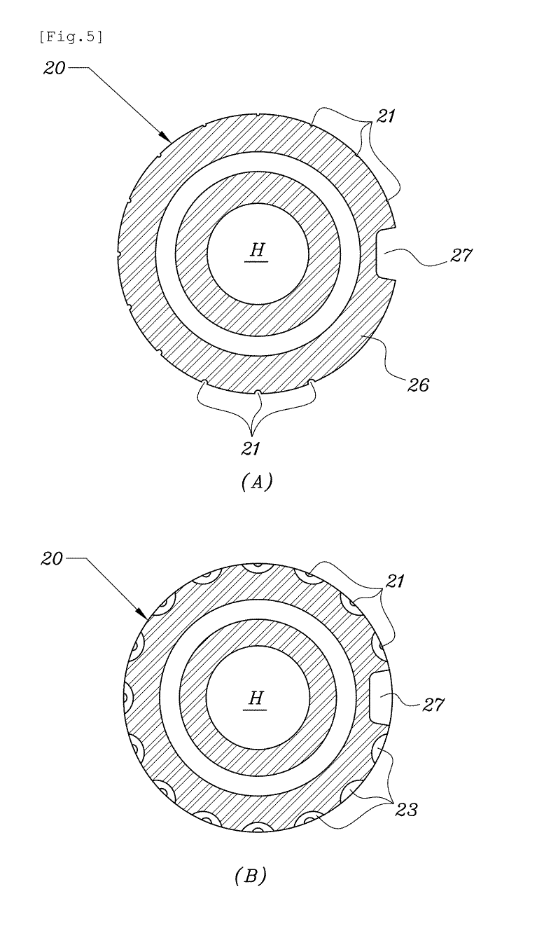

As shown in FIGS. 4 and 5, the flowrate regulating passages 21 disposed on the outer peripheral surface of the rotating member 20 are arranged such that a cross section area of each flowrate regulating passage gradually increases in the peripheral direction of the rotating member 20. Additionally, as the rotating member 20 rotates, one of the flowrate regulating passages 21 is connected to the inflow tube 11.

In addition, the discharge passage 31 formed on the upper surface of the fixing member 30 connects a lower end portion of the flowrate regulating passage 21, which is connected to the inflow tube 11, and the discharge tube 12. Therefore, infusion flowing into the inflow tube 11 passes one of the flowrate regulating passage 21 and the discharge passage 31, and then, is discharged to the discharge tube 12.

In this instance, the cross section area of the flowrate regulating passage 21, which has the greatest cross section area among the flowrate regulating passages 21, does not exceed a cross section area of the discharge passage 31. That is, the cross section area of the flowrate regulating passage 21 does not exceed the cross section area of the discharge passage 31. Therefore, a flowrate of the infusion flowing into the inflow tube 11 is determined by the cross section area of the flowrate regulating passage 21 connected to the inflow tube 11.

Moreover, a user can randomly select the flowrate regulating passage 21 connected to the inflow tube 11 while rotating the rotating member 20. The rotating member 20 has gradations (not shown) indicated on the outer peripheral surface of the rotating member 20 to correspond to the flowrate regulating passage 21 so that the user can easily select his or her wanted flowrate regulating passage 21.

In the meantime, a central hole (H) is vertically perforated in at least one of the rotating member 20 and the fixing member 30 along a central axis line, and an injection port unit 40 may be mounted on the central hole (H). The injection port unit 40 is used for inserting a syringe or a spike, and any one out of general ports for infusion sets may be used. A medical fluid injected through the injection port unit 40 flows into the discharge passage 31 of the fixing member 30 through the central hole (H).

If the injection port unit 40 is mounted at only one of the rotating member 20 and the fixing member 30, it is a three-way structure, and if the injection port units 40 are mounted at the rotating member 20 and the fixing member 30, it is a four-way structure. FIGS. 2 to 4 illustrate the four-way structure that two injection port units 40 are mounted.

FIGS. 3 and 4 illustrate the injection port unit 40 according to the preferred embodiment of the present invention. The injection port unit 40 includes an insertion tube 41 inserted into the central hole (H), a cylindrical port housing 42 mounted at one end portion of the insertion tube 41, a rubber stopper 43 embedded in the port housing 42, and an end cap 44 for preventing separation of the rubber stopper 43.

The rubber stopper 43 has an empty inside and a slit part (S) formed at one side, and the slit part (S) is exposed out of the end cap 44 so that the spike is easily inserted. A filter (not shown) for preventing rubber fragments or foreign matters from entering may be mounted between the insertion tube 41 and the rubber stopper 43. Moreover, an outer cap (not shown) for covering and protecting the slit part (S) of the rubber stopper 43 may be mounted on the end cap 44.

As shown in FIGS. 4 and 5, an angle regulating grooves 23 are respectively mounted at lower end portions of the flowrate regulating passages 21 of the rotating member 20 at regular intervals, and retaining jaws 13 corresponding to the angle regulating grooves 23 are mounted on the inner peripheral surface of the body member 10. So, when the rotating member 20 is rotated, the retaining jaws 13 fix a rotational angle so that one of the flowrate regulating passages 21 is accurately connected to the inflow pipe 11 while getting over the angle regulating grooves 23 one by one.

The infusion flowing into the flowrate regulating passage 21 flows into the discharge passage 31 after passing through the angle regulating grooves 23. Therefore, preferably, the retaining jaws 13 are mounted on the opposite side of the inflow tube 11 as shown in FIG. 4, not to interrupt the flow of the infusion passing through the flowrate regulating passage 21.

Furthermore, as shown in FIG. 2, preferably, a first stopper 14 protrudes from the upper end edge of the body member 10, and a second stopper 24 which corresponds to the first stopper 14 is mounted on the outer peripheral surface of the rotating member 20. The first stopper 14 and the second stopper 24 function to limit a rotational angle of the rotating member 20 to less than 360 degrees.

Additionally, as shown in FIG. 4, an upper annular groove 15 and a lower annular groove 16 are respectively formed on an upper inner peripheral surface and a lower inner peripheral surface of the body member 10 in the circumferential direction, and retaining annular jaws 25 and 32, which respectively combined to the upper annular groove 15 and the lower annular groove 16, are respectively formed on the lower outer peripheral surface of the rotating member and the upper outer peripheral surface of the fixing member 30.

The upper annular groove 15 and the lower annular groove and the retaining annular jaws 25 and 32 respectively corresponding to the annular grooves 15 and 16 can forcedly insert the rotating member 20 and the fixing member 30 into the body member 10. Preferably, the rotating member 20 and the fixing member 30 have a saw-tooth structure that the rotating member 20 and the fixing member 30 correspond to each other not to be separated from the body member 10 once the rotating member 20 and the fixing member 30 are inserted into the body member 10. In this instance, O-rings (not shown) for preventing a leakage may be mounted between the body member 10 and the rotating member 20 and between the body member 10 and the fixing member 30 at proper positions.

In addition, as shown in FIG. 2, a fixing groove 17 is formed at the lower end edge of the body member 10, and a fixing protrusion 33 which is inserted into the fixing groove 17 is disposed on the outer peripheral surface of the fixing member 30. The fixing groove 17 and the fixing protrusion 33 function to fasten the fixing member 30 not to rotate inside the body member 10.

Finally, as shown in FIG. 5, a flow path shield 26 is formed on the outer peripheral surface of the rotating member to interrupt the flow of the infusion by stopping an entrance of the inflow tube 11. The flow path shield 26 is formed at an area, where the flowrate regulating passages 21 are not formed, of the outer peripheral surface of the rotating member 20, and is arranged between the flowrate regulating passage 21, which has the smallest cross section area, and the flowrate regulating passage 21, which has the greatest cross section area.

Moreover, a free pass passage 27 may be mounted next to the flow path shield 26 so that the infusion flowing into the inflow tube 11 can pass without flow regulation. A cross section area of the free pass passage 27 is equal to the cross section areas of the inflow tube 11 and the discharge passage 31.

While the present invention has been particularly shown and described with reference to exemplary embodiments thereof, it will be understood by those of ordinary skill in the art that the scope of the present invention is not limited by the embodiments.

1. A flowrate regulating device for an infusion set comprising:
a body member (10) which is formed in a cylindrical shape opened vertically and has an inflow tube (11) protruding at one side and a discharge tube protruding in the opposite direction from the inflow tube at a position lower than the inflow tube (11);
a rotating member (20) of which the lower part is rotatably combined to the top of the body member (10), the rotating member (20) including a plurality of flowrate regulating passages (21), which are vertically formed on the outer peripheral surface of the lower part getting in contact with the body member (10) side by side at regular intervals, and a rotating lever (22) mounted on the top thereof; and
a fixing member (30) of which the upper part is fixedly combined to the bottom of the body member (10), the fixing member (30) including a discharge passage (31) hollowed on the upper surface getting in contact with the bottom surface of the rotating member (20) in a diameter direction,
wherein the flowrate regulating passages (21) are arranged such that a cross section area of each flowrate regulating passage gradually increases in the peripheral direction of the rotating member (20), and as the rotating member (20) rotates, one of the flowrate regulating passages (21) is connected to the inflow tube (11), the discharge passage (31) is connected between a lower end portion of the flowrate regulating passage (21), which is connected to the inflow tube (11), and the discharge tube (12), and the greatest cross section area of the flowrate regulating passage (21) does not exceed a cross section area of the discharge passage (31).

2. The flowrate regulating device according to claim 1, wherein a central hole (H) is vertically perforated in at least one of the rotating member (20) and the fixing member (30) along a central axis line, and an injection port unit (40) is mounted on the central hole (H).

3. The flowrate regulating device according to claim 2, wherein the injection port unit (40) comprises an insertion tube (41) inserted into the central hole (H), a cylindrical port housing (42) mounted at one end portion of the insertion tube (41), a rubber stopper (43) embedded in the port housing (42) and having a slit part (S) mounted at one side thereof, and an end cap (44) which prevents separation of the rubber stopper 43 and exposes the slit part (S).

4. The flowrate regulating device according to claim 1, wherein an angle regulating grooves (23) are respectively mounted at lower end portions of the flowrate regulating passages (21) at regular intervals, and retaining jaws (13) corresponding to the angle regulating grooves (23) are mounted on the inner peripheral surface of the body member (10), so that the retaining jaws (13) fix a rotational angle to accurately connect one of the flowrate regulating passages (21) to the inflow pipe (11) while getting over the angle regulating grooves (23) one by one when the rotating member (20) is rotated.

5. The flowrate regulating device according to claim 4, wherein the body member (10) is divided into an upper body member (10a) on which the inflow tube (11) is mounted and a lower body member (10b) on which the discharge tube (12) is mounted, and the retaining jaws (13) are formed in the opposite side of the inflow tube (11).

6. The flowrate regulating device according to claim 1, wherein a first stopper (14) protrudes from the upper end edge of the body member (10) and a second stopper (24) which corresponds to the first stopper (14) is mounted on the outer peripheral surface of the rotating member (20), so that the rotational angle of the rotating member (20) is limited less than 360 degrees by the first stopper (14) and the second stopper (24).

7. The flowrate regulating device according to claim 1, wherein an upper annular groove (15) and a lower annular groove (16) are respectively formed on an upper inner peripheral surface and a lower inner peripheral surface of the body member (10) in the circumferential direction, and retaining annular jaws (25, 32), which respectively combined to the upper annular groove (15) and the lower annular groove (16), are respectively formed on the lower outer peripheral surface of the rotating member (20) and the upper outer peripheral surface of the fixing member (30), and
wherein a fixing groove (17) is hollowed at the lower end edge of the body member (10), and a fixing protrusion (33) which is inserted into the fixing groove (17) to limit rotation of the fixing member (30) is disposed on the outer peripheral surface of the fixing member (30).

8. The flowrate regulating device according to claim 1, wherein the rotating member (20) further comprises a flow path shield (26) and a free pass passage (27) on the outer peripheral surface thereof, and the flow path shield (27) interrupts the flow of the infusion by stopping an entrance of the inflow tube (11) between the flowrate regulating passage (21) having the smallest cross sectional area and the flowrate regulating passage (21) having the greatest cross sectional area and the free pass passage (27) makes the infusion flowing into the inflow tube (11) pass without flow regulation.
